(19) United States
(12) Patent Application Publication
(10) Pub. No.: US 2007/0196866 A1
(43) Pub. Date: Aug. 23, 2007

(54) Modulators of ion channel trpa1

(75) Inventors: Ardem Patapoutian, San Diego, CA (US); Michael Bandell, Del Mar, CA (US); Gina M. Story, San Marcos, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignees: IRM LLC, Hamilton (BM); The Scripps Research Institute, La Jolla, CA (US)

(21) Appl. No.: 10/592,648

(22) PCT Filed: Mar. 11, 2005
(86) PCT No.: PCT/US05/08105

§ 371 Date: Sep. 13, 2006

Related U.S. Application Data
(60) Provisional application No. 60/552,860, filed on Mar. 13, 2004.

(51) Int. Cl. G01N 33/567; A61K 31/618; A61K 31/105; A61K 31/11; A61K 31/12

(52) U.S. Cl. 435/7.2; 514/159; 514/701; 514/690; 514/707

(57) ABSTRACT

This invention provides methods for modulating activities of noxious ion channel TRPAI and methods for screening for novel modulators of TRPAI. Compounds such as bradykinin, eugenol, gingerol, methyl salicylate, allicin, and cinnamaldehyde can be employed to activate cold themosensor TRPAI. These TRPAI agonists can be used in screenings methods to activate TRPA1 and therefore identify novel TRPAI antagonists that can inhibit the activated TRPAI. These TRPAI agonists also provide chemical backbones to synthesize and identify analogs with improved biological or pharmaceutical properties. Further, novel TRPAI modulators can be identified by screening test agents for ability in modulating enzymatic activity or cellular level of phospholipase C.

### CROSS-REFERENCE TO RELATED APPLICATIONS

This application claims the benefit of priority under 35 U.S.C. §119(e) to U.S. Provisional Patent Application No. 60/552,860, filed Mar. 13, 2004. The disclosure of the priority application is incorporated herein by reference in its entirety and for all purposes.

### STATEMENT CONCERNING GOVERNMENT SUPPORT

This invention was made in part with government support under NINDS Grant Nos. NS42822 and NS046303 awarded by the National Institutes of Health. The U.S. Government may therefore have certain rights in this invention.

### FIELD OF THE INVENTION

The present invention generally relates to modulation and regulation of an ion channel involved in pain signaling. More particularly, the invention relates to modulators of activities of noxious cold sensor TRPA1, and to industrial and therapeutic applications of such modulators.

### BACKGROUND OF THE INVENTION

Ion channels play a central role in neurobiology as membrane-spanning proteins that regulate the flux of ions. Categorized according to their mechanism of gating, ion channels can be activated by signals such as specific ligands, voltage, or mechanical force. Temperature has been shown to activate certain members of the Transient Receptor Potential (TRP) family of cation channels (Patapoutian et al., Nature Reviews Neuroscience 4, 529-539, 2003). Two members of two distinct subfamilies of TRP channels have been implicated in cold sensation: TRPM8 and TRPA1. TRPM8 is activated at 25° C. It is also the receptor for the compound menthol, providing a molecular explanation of why mint flavors are typically perceived as refreshingly cooling.

TRPA1, also termed ANKTM1, is activated at 17° C. and is a noxious cold-activated ion channel specifically expressed in a subset of TRPV1-, CGRP-, and substance P-expressing nociceptive neurons (Story et al., Cell 112: 819-829, 2003). The TRPA1 ortholog in Drosophila melanogaster also acts as a temperature sensor. Together these temperature-activated channels represent a subset of TRP channels that are dubbed thermoTRPs. In agreement with a role in initiating temperature sensation, most of the thermoTRPs are expressed in subsets of Dorsal Root Ganglia (DRG) neurons that strikingly correlate with the physiological characteristics of thermosensitive DRG neurons. There are neurons that express only TRPV1 (hot), only TRPM8 (cool), or both TRPV1 and TRPA1 (polymodal nociceptors).

Modulation of TRPA1 has numerous industrial and therapeutic applications. For example, there is a need in the art for new analgesic pharmaceutical preparations suitable for the treatment and/or prophylaxis of nociceptive pain in mammals, especially in humans. By providing novel compositions and methods of modulating TRPA1 activities, the present invention fulfills this and other needs.

### SUMMARY OF THE INVENTION

In one aspect, the present invention provides methods for identifying an inhibitor of noxious cold ion channel TRPA1. The methods entail (a) contacting a TRPA1 polypeptide with test agents in the presence of a TRPA1 agonist; and (b) identifying a modulating agent that suppresses or reduces a signaling activity of the TRPA1 polypeptide relative to the activity of the TRPA1 polypeptide in the absence of the test agent. The TRPA1 agonist to be used in these methods is selected from the group consisting of allicin, cinnamaldehyde, eugenol, gingerol, and methyl salicylate. In some methods, the TRPA1 agonist is put into contact with the TRPA1 polypeptide prior to contacting the TRPA1 polypeptide with the test agents. In some methods, the TRPA1 polypeptide employed is human TRPA1 or mouse TRPA1.

In some of these methods, the TRPA1 polypeptide is present in a TRPA1-expressing cell or a cultured neuron. In some methods, the cultured neuron is a cultured DRG neuron. In some methods, the cell is a TRPA1-expressing CHO cell or a TRPA1-expressing Xenopus oocyte. In some of these methods, the signaling activity is calcium influx into the TRPA1-expressing cell or the cultured neuron. In some other methods, the signaling activity is increased intracellular free calcium level of the TRPA1-expressing cell or the cultured neuron.

In another aspect, the invention provides methods for identifying an agent that modulates noxious cold ion channel TRPA1. The methods involve (a) assaying a biological activity of a phospholipase C (PLC) polypeptide in the presence of a test agent to identify one or more modulating agents that modulate the biological activity of the PLC polypeptide; and (b) testing one or more of the modulating agents for ability to modulate an activity mediated by TRPA1. In some of these methods, the PLC polypeptide employed is a PLC isoform that is expressed in dorsal root ganglia (DRG) neurons that express TRPA1. In some methods, the modulating agents inhibit the activity mediated by TRPA1. In some other methods, the modulating agents activate the activity mediated by TRPA1.

In some of these methods, the modulating agents identified are tested for ability to modulate calcium influx of a TRPA1-expressing cell. For example, the cell can be a TRPA1-expressing CHO cell or a TRPA1-expressing Xenopus oocyte. The cell can also be a cultured DRG neuron that expresses TRPA1. Some of the cells used in the methods stably express TRPA1. In some methods, the TRPA1 employed is human TRPA1 or mouse TRPA1. The biological activity assayed in the methods can be a binding to the test agents by the PLC polypeptide, cellular level of the PLC polypeptide, or an enzymatic activity of the PLC polypeptide (e.g., catalyzing breakdown of PIP2 into DAG and IP3).

In one aspect, the invention provides methods for identifying a TRPA1 activator with improved properties over that of a TRPA1 agonist described herein. The methods involve (a) synthesizing one or more structural analogs of a TRPA1 agonist; and (b) performing a functional assay on the analogs to identify an analog that has an improved biological or pharmaceutical property relative to that of the TRPA1 agonist. The TRPA1 agonist employed in these methods is selected from the group consisting of allicin, cinnamaldehyde, eugenol, gingerol, and methyl salicylate. In some of these methods, the improved biological or pharmaceutical property is an enhanced binding affinity for TRPA1. In some other methods, the improved biological or pharmaceutical property is an increased ability to penetrate the skins.

In another aspect, the invention provides methods for stimulating sensory perception in a subject. The methods entail (a) providing a subject that contains noxious cold-activated ion channel TRPA1, and (b) administering to the subject a pharmaceutical composition comprising an effective amount of a compound selected from the group consisting of eugenol, gingerol, methyl salicylate, allicin, and cinnamaldehyde. In some methods, the compound is administered to the subject as a food additive.

In another aspect, the invention provides methods for reducing nociceptive pain in a subject. These methods involve (a) providing a subject expressing TRPA1, and (b) administering to the subject a pharmaceutical composition comprising an effective amount of U-73 122.

A further understanding of the nature and advantages of the present invention may be realized by reference to the remaining portions of the specification and claims.

### DETAILED DESCRIPTION

In human and other vertebrates, painful stimuli and noxious temperature are sensed by specialized neurons known as nociceptors. These molecules fire in response to noxious temperature and mechanical or chemical stimuli, all of which have the potential to cause tissue damage. The signals are in turn processed by the central nervous system and perceived as pain, serving an indispensable protective role. Nociceptors are also involved in pathological pain states caused by inflammation, nerve damage, or cancer.

The present invention is predicated in part on the discovery that TRPA1 is modulated (activated or inhibited) by a variety of noxious molecules. The present inventors further discovered that activation of TRPA1 is an important component of pain sensation that signals the noxious, burning element of cold. In accordance with these discoveries, the present invention provides novel compounds that modulate TRPA1 activities and methods relating to therapeutic and prophylactic applications of such compounds.

The following sections provide guidance for making and using the compositions of the invention, and for carrying out the methods of the invention.

I. Definitions

Unless defined otherwise, all technical and scientific terms used herein have the same meaning as commonly understood by those of ordinary skill in the art to which this invention pertains. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY (1991). In addition, the following definitions are provided to assist the reader in the practice of the invention.

The term “agent” or “test agent” includes any substance, molecule, element, compound, entity, or a combination thereof. It includes, but is not limited to, e.g., protein, polypeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” are used interchangeably herein.

The term “analog” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved traits (such as higher binding affinity for a target molecule) is an approach that is well known in pharmaceutical chemistry.

“Antinociception” means abatement or inhibition of acute or chronic nociceptive pain. Pain perception is transmitted by nociceptors, specialized nerve fibers.

As used herein, “contacting” has its normal meaning and refers to combining two or more agents (e.g., polypeptides or small molecule compounds) or combining agents and cells. Contacting can occur in vitro, e.g., combining two or more agents or combining a test agent and a cell or a cell lysate in a test tube or other container. Contacting can also occur in a cell or in situ, e.g., contacting two polypeptides in a cell by coexpression in the cell of recombinant polynucleotides encoding the two polypeptides, or in a cell lysate.

As used herein, “hyperalgesia” or a “hyperalgesic state” refers to a condition in which a warm-blooded animal is extremely sensitive to mechanical, chemical or thermal stimulation that, absent the condition, would be painless. Typical models for such a hyperalgesic state include the inflamed rat paw compression model and the compression of the inflamed knee joint.

Hyperalgesia is known to accompany certain physical injuries to the body, for example the injury inevitably caused by surgery. Hyperalgesia is also known to accompany certain inflammatory conditions in man such as arthritic and rheumatic disease. Hyperalgesia thus refers to mild to moderate pain to severe pain such as the pain associated with, but not limited to, inflammatory conditions (e.g., such as rheumatoid arthritis and osteoarthritis), postoperative pain, post-partum pain, the pain associated with dental conditions (e.g., dental caries and gingivitis), the pain associated with burns, including but not limited to sunburns, abrasions, contusions and the like, the pain associated with sports injuries and sprains, inflammatory skin conditions, including but not limited to poison ivy, and allergic rashes and dermatitis, and other pains that increase sensitivity to mild stimuli, such as noxious cold.

A “heterologous sequence” or a “heterologous nucleic acid,” as used herein, is one that originates from a source foreign to the particular host cell, or, if from the same source, is modified from its original form. Thus, a heterologous gene in a host cell includes a gene that, although being endogenous to the particular host cell, has been modified. Modification of the heterologous sequence can occur, e.g., by treating the DNA with a restriction enzyme to generate a DNA fragment that is capable of being operably linked to the promoter. Techniques such as site-directed mutagenesis are also useful for modifying a heterologous nucleic acid.

The terms “homology” and “identity” in the context of two or more nucleic acids or polypeptide sequences, refer to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “host cell,” as used herein, refers to a prokaryotic or eukaryotic cell into which a heterologous polynucleotide can be or has been introduced. The heterologous polynucleotide can be introduced into the cell by any means, e.g., electroporation, calcium phosphate precipitation, microinjection, transformation, viral infection, and/or the like.

The term “modulate” with respect to a reference protein (e.g., a TRPA1 or a PLC polypeptide) refers to inhibition or activation of a biological activity of the reference protein (e.g., a pain signaling related activity of TRPA1). Modulation can be up-regulation (i.e., activation or stimulation) or down-regulation (i.e., inhibition or suppression). The mode of action can be direct, e.g., through binding to the reference protein as a ligand. The modulation can also be indirect, e.g., through binding to and/or modifying another molecule which otherwise binds to and modulates the reference protein.

“Polynucleotide” or “nucleic acid sequence” refers to a polymeric form of nucleotides (polyribonucleotide or polydeoxyribonucleotide). In some instances a polynucleotide refers to a sequence that is not immediately contiguous with either of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (e.g., a cDNA) independent of other sequences. Polynucleotides can be ribonucleotides, deoxyribonucleotides, or modified forms of either nucleotide.

A polypeptide or protein (e.g., TRPA1) refers to a polymer in which the monomers are amino acid residues that are joined together through amide bonds. When the amino acids are alpha-amino acids, either the L-optical isomer or the D-optical isomer can be used, the L-isomers being typical. A polypeptide or protein fragment (e.g., of TRPA1) can have the same or substantially identical amino acid sequence as the naturally occurring protein. A polypeptide or peptide having substantially identical sequence means that an amino acid sequence is largely, but not entirely, the same, but retains a functional activity of the sequence to which it is related.

Polypeptides may be substantially related due to conservative substitutions, e.g., TRPA1 and a TRPA1 variant containing such substitutions. A conservative variation denotes the replacement of an amino acid residue by another, biologically similar residue. Examples of conservative variations include the substitution of one hydrophobic residue such as isoleucine, valine, leucine or methionine for another, or the substitution of one polar residue for another, such as the substitution of arginine for lysine, glutamic for aspartic acids, or glutamine for asparagine, and the like. Other illustrative examples of conservative substitutions include the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine to leucine.

A “substantially pure polypeptide” is typically pure when it is at least 60%, at least 75%, more preferably at least 90%, and most preferably at least 99%, by weight, free from the proteins and naturally occurring organic molecules with which it is naturally associated. A substantially pure polypeptide (e.g., a TRPA1 polypeptide) may be obtained, for example, by extraction from a natural source (e.g., a mammalian cell); by expression of a recombinant nucleic acid encoding the polypeptide; or by chemically synthesizing the polypeptide. Purity can be measured by any appropriate method, e.g., by column chromatography, polyacrylamide gel electrophoresis, or by HPLC analysis.

As used herein, the phrase “screening for TRPA1 modulators” refers to use of an appropriate assay system to identify novel TRPA1 modulators from test agents. The assay can be an in vitro or an in vivo assay suitable for identifying whether a test agent can stimulate or suppress one or more of the biological functions of a TRPA1 molecule or a phospholipase C (PLC) polypeptide. Examples of suitable bioassays include, but are not limited to, assays for examining binding of test agents to a PLC polypeptide or a TRPA1 polypeptide (e.g., a TRPA1 fragment containing its ligand binding domain), calcium influx assay, or behavior analysis. Either an intact PLC or TRPA1 polypeptide or polynucleotide, fragments, variants, or substantially identical sequences may be used in the screening.

The term “subject” includes mammals, especially humans, as well as other non-human animals, e.g., horse, dogs and cats.

A “variant” of a reference molecule (e.g., a TRPA1 polypeptide or a TRPA1 modulator) is meant to refer to a molecule substantially similar in structure and biological activity to either the entire reference molecule, or to a fragment thereof. Thus, provided that two molecules possess a similar activity, they are considered variants as that term is used herein even if the composition or secondary, tertiary, or quaternary structure of one of the molecules is not identical to that found in the other, or if the sequence of amino acid residues is not identical.

II. TRPA1 and TRPA1-Modulating Compounds

TRPA1 belongs to the superfamily of TRP channels as does the menthol- and cold-activated receptor, TRPM8, despite the lack of amino acid sequence similarity between the two. Like other thermosensitive TRPs, TRPA1 is a non-selective cation channel. Human and mouse TRPA1 sequences are known. Theoretical translation of the mouse nucleotide sequence predicts a protein of 1125 amino acid residues, while human TRPA1 has 1119 amino acids.

Both TRPM8 and TRPA1 respond to cold. However, TRPA1 displays several unique characteristics compared to previously characterized temperature-activated TRP channels. The variability in activation threshold temperature of TRPA1 from cell to cell is broader when compared to other TRPs. Furthermore, the current through TRPA1 rapidly desensitizes to cold, a property not seen to such an extent in other temperature-activated TRPs. Finally, long-term overexpression of TRPA1 is detrimental to cells, making it necessary for cell lines to conditionally express TRPA1.

Human and mouse TRPA1 ion channels are activated by noxious cold temperatures. TRPA1 is activated at lower temperatures than TRPM8, starting at near 17° C., which approximates the threshold of noxious cold for humans (˜15° C.). Mouse TRPA1 is specifically expressed in somatic sensory neurons. Within this population, TRPA1 is not expressed in neurons that express TRPM8. Instead, the vast majority of TRPA1-positive cells also express TRPV1 and CGRP, markers for pain-sensing neurons. There are likely two separate populations of cold-sensitive DRG neurons: one population that expresses TRPM8 and is menthol-sensitive, and a distinct population that is menthol-insensitive and is activated at even colder temperatures. It is likely that TRPA1 marks this second population of cold sensitive neurons.

The present inventors also observed that TRPA1 is activated by an algogenic peptide and a variety of natural pungent compounds present in foods and flavoring products. First, as detailed in the Examples below, it was found that cinnamaldehyde, a specific TRPA1 activator in vitro, predominantly excites cold-sensitive DRG neurons in culture. The response profile of menthol and cinnamaldehyde accurately reflect the mutually exclusive expression of the two cold-activated ion channels TRPM8 and TRPA1, respectively. Indeed, cinnamaldehyde- and menthol-responding neurons account for almost all cold-responsive neurons in culture (32/33). In addition, it was found that external Ca²⁺ dramatically augments cold-induced activation of TRPA1 but is not required for cinnamaldehyde-induced activation.

Further studies indicate that TRPA1 is activated by cinnamaldehyde and other sensory compounds. These include a variety of pungent compounds - oils of cinnamon, allicin from fresh garlic, mustard, wintergreen, ginger, and clove, which all activate TRPA1. Cinnamaldehyde is the main constituent of cinnamon oil (˜70%) and is extensively used for flavoring purposes in foods, chewing gums, and toothpastes. Allyl isothyocianate (mustard oil) is one of the active ingredients in horseradish and wasabi. Methyl Salicylate (wintergreen oil) is used commonly in products such as Listerine, IcyHot, and Bengay for its burning effect. The specificity of these TRPA1-activating compounds was tested against other thermoTRPs. Cinnamaldehyde and allyl isothyocianate activate only TRPA1. Moreover, cinnamaldehyde preferentially activates a subset of cold-activated cultured adult DRG neurons that have TRPA1-like profile. Mustard oil activates this same population, in addition to a larger cold-insensitive group of neurons.

Cinnamaldehyde activates TRPA1-expressing CHO cells in micromolar concentrations, and TRPA1 is expressed in trigeminal neurons that project to the tongue. Therefore, TRPA1 could be responsible for the burning sensory quality of cinnameldehyde. Traditionally, the gustatory and olfactory systems are thought to account for the perception of oral flavorings. The extended list of sensory compounds that activate thermoTRPs provides molecular evidence that the trigeminal system also plays an important role in taste perception.

To study sensory quality of compounds from rodents, mice were intraplantarly injected with cinnamaldehyde. As detailed below, the results showed that cinnamaldehyde causes noxious response behavior and thermal hyperalgesia. The data indicates that cinnamaldehyde could activate nociceptive neurons, consistent with TRPA1 expression in CGRP- and substance P-expressing neurons.

In addition to the above-noted pungent compounds, it was also found that TRPA1 is activated by an algogenic inflammatory peptide the bradykinin (BK). The activation of many TRP ion channels is linked to G protein coupled receptor (GPCR) signaling. The present inventors found that TRPA1 can be activated by BK, an inflammatory signal involved in nociception that acts through its GPCR. BK directly excites nociceptive DRG neurons and causes hyperalgesia. Mechanisms of BK-induced hyperalgesia are well studied; however, the identity of the ion channels acutely activated by BK is not known. The electrophysiological data as detailed in the Examples below indicate that TRPA1 is coupled to the activation of the BK2 receptor. It was also shown that majority of cinnamaldehyde-responding neurons are also activated by BK in adult DRG cultures. These observations indicate that TRPA1 is an endogenous component of BK-induced excitation of polymodal nociceptors.

III. Industrial Applications

The novel TRAPA1-activating agents of the present inventors can have various industrial applications. These include the TRPA1-activating compounds described above, as well as other TRPA1-stimulating modulators that can be identified in accordance with the present invention. By activating TRPA1, these compounds, e.g., allicin, eugenol, gingerol, methyl salicylate, allyl isothiocyanate and cinnamaldehyde, can stimulate sensory perception by a subject. This could have many practical utilities. For example, these compounds can be used as flavoring or refreshing agents in various compositions, articles or products.

By enhancing sensations, the TRPA1-activating compounds can be used as food additives to enhance flavors of various foodstuffs to which they are added. Flavoring agents, individually or in combination, are used to impart desired flavor characteristics to a variety of consumable products. The TRPA1-activating compounds of the present invention can be used alone or in combination with other flavoring agents in order to provide interesting and pleasing flavor perceptions. For example, any of the TRPA1-activating compounds disclosed herein can be used together with flavoring agents such as corn mint oil, cardamom, and menthol.

In addition to food industry, these TRPA1-activating compounds can also be used in other fields where enhanced sensory perception is desired. For example, the TRPA1-activating compounds can find applications in body-care or cosmetic products. In general, these compounds can be used in all fields in which a cooling effect is to be imparted to the products in which they are incorporated. By way of example one may cite beverages such as fruit juices, soft drinks or cold tea, ice creams and sorbets, sweets, confectioneries, chewing gum, chewing tobacco, cigarettes, pharmaceutical preparations, dental-care products such as dentifrice gels and pastes, mouth washes, gargles, body and hair care products such as shampoos, shower or bath gels, body deodorants and antiperspirants, after-shave lotions and balms, shaving foams, perfumes, etc.

As noted in the Examples below, all these TRPA1-activating compounds are readily available from commercial sources. In addition, methods of incorporating flavoring or refreshing agents into consumer products are well known in the art, e.g., as described in U.S. Pat. No. 6,359,168. In general, the proportions in which the TRPA1-activating compounds of the invention may be incorporated into the various products mentioned above vary within a wide range of values. These values depend on the nature of the article or product to which a cooling effect is to be imparted and on the effect required. They also depend on the nature of the co-ingredients in a given composition when the compounds of the invention are used in a mixture with flavoring or perfuming co-ingredients, solvents or adjuvants commonly used in the art.

Typically, the concentration of a TRPA1-activating compound is in the order of 0.001 to 5% or more, preferably 0.002 to 1%, by weight of the compound of the present invention relative to the finished product in which it is incorporated. For example, in applications such as beverages and sweets, concentrations of the order of 0.005 to 0.1% will typically be used. In comparison, for flavoring dentifrices and chewing gums, the compounds of the invention will typically be used in concentrations within the range 0.2-0.3 and 0.5-1%.

IV. Screening for Novel TRPA1 Modulators

In addition to the TRPA1-modulating compounds described herein, the invention also provides methods of screening for novel TRPA1 modulators.

A. Screening Methods Using Novel TRPA1 Agonists of the Present Invention

The invention provides screening methods for identifying TRPA1 modulators, utilizing the novel TRPA1 agonists identified by the present inventors. These methods are particularly suitable for identifying novel inhibitory modulators of TRPA1, preferably in a high throughput format. TRPA1 is normally not active. To identify novel TRPA1 antagonists in a screen assay, TRPA1 must be activated first. One way to accomplish this is to apply cold. However, this approach is not practical in a high throughout screening format. The TRPAI agonists (e.g., cinnamaldehyde) identified by the present inventors provide novel means for activating TRPA1 in order to screen for compounds that will inhibit or suppress activities of the activated TRPA1.

Typically, these methods involve contacting a TRPA1 polypeptide with test agents in the presence of a TRPA1 agonist described herein. The TRPA1 agonist (e.g., cinnamaldehyde, allicin, eugenol, gingerol, or methyl salicylate) can be added to a cell-expressing TRPA1 before, concurrently with, or after contacting the cell with test agents. If a test agent suppresses or inhibits an activity of the activated TRPA1 (e.g., a noxious cold related pain signaling activity described below), a novel TRPA1 antagonist or inhibitor is identified. In some methods, instead of employing a cell expressing TRPA1, a TRPA1 polypeptide can be used. TRPA1 antagonists may be identified from test agents that inhibit an activity of the TRPA1 polypeptide (e.g., a biochemical property) after contacting the TRPA1 polypeptide with a TRPA1 agonist (e.g., cinnamaldehyde). Preferably, these screening methods are performed in a high throughput format. For example, each test agent can be put into contact with a TRPA1-expressing cell in a different well of a microtiter plate. The TRPA1 agonist is present in each of these wells to activate TRPA1.

B. Screening Novel TRPA1 Modulators Using PLC

Some other screening methods of the invention are based in part on the discovery by the present inventors that phospholipase C is required for TRPA1 activation. As noted above, TRPA1 is activated by cold, a variety of pungent compounds, and bradykinin. Additional observations as detailed in the Examples below show that activation by any of these stimuli is severely attenuated by a specific phospholipase C (PLC) inhibitor. One of the consequences of PLC activation is breakdown of phosphatidylinositol-4,5-bisphosphate (PIP2) into diacylglycerol (DAG) and inositol triphosphate (IP3). Cinnamaldehyde and cold do not cause a release of calcium from cells not expressing TRPA1, and therefore it is unlikely that these stimuli activate TRPA1 through PLC activation. Instead, the data indicates that basal PLC activity is required for proper function of this channel. TRPA1 might require basal PLC activity to keep the channel in a state that is primed for activation. In addition, the data indicates that robust PLC activation (for example, via BK2R) can be sufficient to gate TRPA1, perhaps via DAG or arachidonic acid (AA).

Accordingly, the present invention provides novel PLC-based screening methods for identifying novel agents that can modulate TRPA1 activities. These methods involve screening from test agents for modulators of PLC activities using an appropriate assay system. The assay can be an in vitro or an in vivo assay suitable for identifying whether a test agent can inhibit or stimulate the enzymatic functions of PLC. Some of these methods are directed to identifying TRPA1 inhibitors by screening test agents for compounds that inhibit PLC activities. In some of these methods which utilize a high throughput format, a known TRPA1 agonist (e.g., cinnamaldehyde) is typically present in order to first activate TRPA1 as described above.

In some of the PLC based screening methods, the PLC polypeptide employed is the PLC isoform that is expressed in dorsal root ganglia (DRG) neurons that express TRPA1. Such methods can enable identification of TRPA1 modulators that specifically inhibit PLC activities in TRPA1-expressing neurons, but not other PLC isoforms that are expressed in other type of cells. These TRPA1-specific PLC inhibitors are therapeutically useful for blocking sensory perception of pain.

Using standard biochemical and molecular biology techniques (e.g., methods described in Sambrook et al., supra; and Ausubel et al., supra), one of ordinary skills in the art could easily identify and ascertain the specific PLC isoform that is expressed in TRPA1-expressing DRG neurons. PLC polynucleotide and amino acid sequences from various species (e.g., human and mouse) are all well known in the art. Their structures and functional organizations, including their ligand binding domains, have also been characterized in the art. See, e.g., Takahashi et al., Methods Enzymol. 71: 710-25, 1981; Hostetler et al., Biochem Biophys Res Commun. 96: 388-93, 1980. For example, polynucleotide sequences encoding various human PLC variants are known in the art, e.g., NM_{—}002660, NM_{—}182811, NM_{—}032726, BC011772, BC006355, BC018646, BC014561, NM_{—}182797, NM_{—}000933, NM_{—}015192, NM_{—}182734, BC050382, and BC041625. Sequences encoding PLC from various other species are also known, e.g., NM_{—}152813, BC065091, BC057161, NM_{—}174425, NM_{—}053758, NM_{—}024353, NM_{—}057503, and NM_{—}057504. Any of these sequences can be used to identify and obtain the PLC polynucleotide and/or polynucleotide that are naturally present in TRPA1-expressing neurons.

C. Screening Schemes

A number of assay systems can be employed in the above-described screening methods to identify novel TRPA1 modulators. Examples of suitable bioassays to screening test agents for modulators of PLC include, but are not limited to, assays for examining binding of test agents to a PLC polypeptide or for measuring PLC activity in converting phosphatidylinositol-4,5-bisphosphate (PIP2) into diacylglycerol (DAG) and inositol triphosphate (IP3). In some methods, test agents are first assayed for their ability to modulate a biological activity of a PLC polypeptide (“the first assay step”). Modulating agents thus identified are then subject to further screening for ability to modulate TRPA1 activities, typically in the presence of the PLC polypeptide (“the second testing step”).

Either an intact PLC polypeptide and TRPA1 or their fragments, analogs, or functional derivatives can be used in these screening methods. The fragments that can be employed in these assays usually retain one or more of the biological activities of the PLC polypeptide (e.g., its enzymatic activity) and TRPA1. Variants, fragments, or functional derivatives of these polypeptides can be prepared from a naturally occurring or recombinantly expressed PLC polypeptide or TRPA1 by proteolytic cleavage followed by conventional purification procedures known to those skilled in the art. Alternatively, they can be produced by recombinant DNA technology by expressing only fragments of a PLC polypeptide or a TRPA1 polypeptide that retain one or more of their bioactivities.

Test agents that can be screened for novel TRPA1 modulators (e.g., inhibitors) include polypeptides, beta-turn mimetics, polysaccharides, phospholipids, hormones, prostaglandins, steroids, aromatic compounds, heterocyclic compounds, benzodiazepines, oligomeric N-substituted glycines, oligocarbamates, polypeptides, saccharides, fatty acids, steroids, purines, pyrimidines, derivatives, structural analogs or combinations thereof. Some test agents are synthetic molecules, and others natural molecules.

Test agents are obtained from a wide variety of sources including libraries of synthetic or natural compounds. Combinatorial libraries can be produced for many types of compound that can be synthesized in a step-by-step fashion. Large combinatorial libraries of compounds can be constructed by the encoded synthetic libraries (ESL) method described in WO 95/12608, WO 93/06121, WO 94/08051, WO 95/35503 and WO 95/30642. Peptide libraries can also be generated by phage display methods (see, e.g., Devlin, WO 91/18980). Libraries of natural compounds in the form of bacterial, fungal, plant and animal extracts can be obtained from commercial sources or collected in the field. Known pharmacological agents can be subject to directed or random chemical modifications, such as acylation, alkylation, esterification, amidification to produce structural analogs.

In some preferred methods, the test agents are small organic molecules (e.g., molecules with a molecular weight of not more than about 1,000). Preferably, high throughput assays are adapted and used to screen for such small molecules. In some methods, combinatorial libraries of small molecule test agents can be readily employed to screen for small molecule modulators of TRPA1. A number of assays are available for such screening, e.g., as described in Schultz et al., Bioorg Med Chem Lett 8: 2409-2414, 1998; Weller et al., Mol. Divers. 3: 61-70, 1997; Fernandes et al., Curr Opin Chem Biol 2: 597-603, 1998; and Sittampalam et al., Curr Opin Chem Biol 1: 384-91, 1997.

Typically, in the PLC based screening methods, test agents are first screened for ability to modulate a biological activity of the PLC polypeptide. In some of these methods, test agents are assayed for specific binding to the PLC polypeptide. Agents thus identified can then be further tested for its ability to alter the enzymatic activity of the PLC polypeptide. Many assays well known in the art can be employed to screen for agents that bind to PLC. These include, e.g., mobility shift DNA-binding assays, methylation and uracil interference assays, DNase and hydroxy radical footprinting analysis, fluorescence polarization, and UV crosslinking or chemical cross-linkers. For a general overview, see, e.g., Sambrook et al., Molecular Cloning: A Laboratory Manual, Cold Spring Harbor Press, N.Y., 3^{rd} Ed. (2000); Ausubel et al., Current Protocols in Molecular Biology, John Wiley & Sons, Inc., New York (1999); and Berger and Kimmel, Methods In Enzymology, San Diego, Academic Press, Inc. (1987).

In some preferred embodiments, the test agents are directly assayed for ability to modulate the enzymatic activity of a PLC polypeptide without assaying their binding to the PLC polypeptide first. Methods for measuring the enzymatic activity of PLC are well known and routinely practiced in the art. See, e.g., Krug et al., Methods Enzymol. 72: 347-51, 1981; De Silva et al., J Clin Microbiol. 25: 729-31, 1987; Hill et al., Anticancer Drug Des. 9: 353-61, 1994; O'Neill et al., Brain Res. 543: 307-14, 1991; and Myung et al., Anal Biochem. 270: 303-13, 1999.

Other than screening for binding to a PLC polypeptide or for activity in modulating its enzymatic function, test agents can also be screened for other activities in the first assay step. For example, they can be assayed for ability to modulate expression level of the PLC polypeptide, e.g., at transcription or translation level. The test agents can also be assayed for activities in modulating cellular level or stability of the PLC polypeptide, e.g., post-translational modification or proteolysis. Expression or cellular level of a PLC polypeptide can be monitored with a number of assays well known and routinely practiced in the art. For example, in a typical cell based assay, a construct comprising a PLC transcription regulatory element operably linked to a reporter gene is introduced into a host cell system. The activity of a polypeptide encoded by the reporter gene (i.e., reporter polypeptide), e.g., an enzymatic activity, in the presence of a test agent can be determined and compared to the activity of the reporter polypeptide in the absence of the test agent. The reporter gene can encode any detectable polypeptide known in the art, e.g., detectable by fluorescence or phosphorescence or by virtue of its possessing an enzymatic activity. The detectable reporter polypeptide can be, e.g., luciferase, alpha-glucuronidase, alpha-galactosidase, chloramphenicol acetyl transferase, green fluorescent protein, enhanced green fluorescent protein, and the human secreted alkaline phosphatase.

Optionally, test agents that modulate (inhibiting or stimulating) the enzymatic activity or cellular level of a PLC polypeptide can be then further examined for ability to modulate a signaling activity of TRPA1 in a second testing step. This assay serves to confirm that the modulating agents identified from the first assay step can indeed modulate TRPA1 signaling activity. Similar assays can be employed in the above-described screening methods of the present invention that utilizing a TRPAI agonist. For example, in these screening methods, test agents can be screened for ability to inhibit or suppress a signaling activity of a TRPA1 polypeptide that has been activated by the TRPA1 agonist.

Ability of a modulating agent or a test agent to modulate TRPA1 signaling activities can be monitored by contacting a TRPA1-expressing cell with the agent, and detecting a decrease or increase in a signaling activity of the cell relative to the activity of the cell in the absence of the test agent. Any activities of TRPA1 that are related to sensory perception of noxious cold or pain (as described in the Examples below) can be monitored in this screening step. For example, the agents can be tested for ability to modulate calcium influx or intracellular free calcium level of a TRPA1-expressing cell or a cultured neuron. They can be assayed for activity in modulating whole-cell membrane currents of TRPA1-expressing cells. They can also be examined for ability to modulate TRPA1 activity in a behavior assay. For example, as exemplified in the Examples below, a TRPA1-modulating activity may be monitored in a paw withdrawal latency test.

D. Analogs of TRPA1 Agonists with Improved Properties

Some of the screening methods of the present invention are directed to identifying analogs or derivatives of the above-described TRPA1 agonists with improved properties. An important step in the drug discovery process is the selection of a suitable lead chemical template upon which to base a chemistry analog program. The process of identifying a lead chemical template for a given molecular target typically involves screening a large number of compounds (often more than 100,000) in a functional assay, selecting a subset based on some arbitrary activity threshold for testing in a secondary assay to confirm activity, and then assessing the remaining active compounds for suitability of chemical elaboration.

The novel TRPA1 agonists described herein, e.g., cinnamaldehyde or allicin, provide lead compounds to search for related compounds that have improved biological or pharmaceutical properties. For example, analogs or derivatives of these TRPA1 agonists can be screened for to identify compounds that have a higher affinity to TRPA1 or are more penetrant of the skin. Compounds with such improved properties can be more suitable for various pharmaceutical applications. For instance, cinnamaldehyde is poorly absorbed through skin. Cinnamaldehyde analogs which can better penetrate the skins will be more useful in some of the industrial and therapeutic applications of the present invention.

These methods typically involve synthesizing analogs, derivatives or variants of a TRPA1 agonist (e.g., allicin, eugenol, gingerol, methyl salicylate, or cinnamaldehyde). Often, a library of structural analogs of a given TRPA1 agonist is prepared for the screening. A functional assay is then performed to identify one or of the analogs or derivatives that have an improved biological property relative to that of the TRPA1 agonist from which the analogs or variants are derived. As noted above, the analogs can be screened for enhanced binding affinity for a TRPA1 polypeptide. Alternatively, they can be assayed to identify compounds with better pharmaceutical properties, e.g., skin penetration or pharmacokinetic characters.

Structures and chemical properties of these TRPA1 agonists (e.g., allicin, eugenol, gingerol, methyl salicylate, or cinnamaldehyde) are all well known and characterized in the art. To synthesize analogs or derivatives based from the chemical backbones of these TRPA1 activators, only routinely practiced methods of organic chemistry that are well known to one of ordinary skill in the art are required. For example, combinatorial libraries of chemical analogs of a known compound can be produced using methods described above. Exemplary methods for synthesizing analogs of various compounds are described in, e.g., by Overman, Organic Reactions, Volumes 1-62, Wiley-Interscience (2003); Broom et al., Fed Proc. 45: 2779-83, 1986; Ben-Menahem et al., Recent Prog Horm Res. 54:271-88, 1999; Schramm et al., Annu. Rev. Biochem. 67: 693-720, 1998; Bolin et al., Biopolymers 37: 57-66, 1995; Karten et al., Endocr Rev. 7: 44-66, 1986; Ho et al., Tactics of Organic Synthesis, Wiley-Interscience; (1994); and Scheit et al., Nucleotide Analogs: Synthesis and Biological Function, John Wiley & Sons (1980).

In addition, any of the above-described assays (e.g., binding assays) can be used to identify an improved property (e.g., enhanced binding affinity for TRPA1) in analogs or derivatives of a given TRPA1 agonist. Additional biochemical or pharmaceutical assays that can be employed are also well known and routinely practiced in the art. For example, skin penetration of a cinnamaldehyde analog can be assayed using methods such as those described in, e.g., Remington's Pharmaceutical Sciences, 18th ed., Mack Publishing Co. (1990).

V. Therapeutic Applications

TRPA1 modulators identified by the present inventors also find therapeutic or prophylactic (e.g., antinociceptive) applications. Accordingly, the invention provides methods for inducing analgesia or reducing pain sensation or perception in a subject. These methods can be used to treat or ameliorate symptoms of a disorder associated with nociception, such as hyperalgesia and nociceptive pain associated disorders. By inhibiting TRPA1 mediated nociception, certain pain perceptions of the subject can be reduced or inhibited.

Various nociceptive pains are suitable for treatment with methods of the invention. Nociceptive pain includes all forms of somatic pain which result from damage or dysfunction of non-neural tissue. Acute nociceptive pain includes pain resulting from tissue-damaging stimulation such as that produced by injury or disease. Examples include postoperative pain, post traumatic pain, acute pancreatis, labor pain, muscle pain and pain accompanying myocardial infarction. Chronic nociceptive pain includes inflammatory pain, arthritis pain, cancer pain and other forms of persistent pain deriving from damaged or inflamed somatic tissue.

Generally, the treatment should affect a subject, tissue or cell to obtain a desired pharmacologic and/or physiologic effect. The effect may be prophylactic in terms of completely or partially preventing a disease or sign or symptom thereof. It can also be therapeutic in terms of a partial or complete cure for hyperalgesia and nociceptive pain associated disorders and/or adverse effect (e.g., pain) that is attributable to the disorders. Suitable subjects include an invertebrate, a vertebrate, a mammal, particularly a human.

The therapeutic methods of the invention entail administering to a subject a pharmaceutical composition that comprises an effective amount of a TRPA1-inhibiting agent of the invention (e.g., U-73122 or a derivative thereof, as exemplified in the Examples below). Novel TRPA1 inhibitors that can be identified in accordance with the screening methods of the invention can also be employed. Administering the pharmaceutical composition may be accomplished by any means known to the skilled artisan. Preferably a subject is a mammal, e.g., a human or a non-human mammal, but may be any other organism that expresses TRPA1. The TRPA1-inhibiting compounds of the present invention can be used alone or in conjunction with other known analgesic agents to alleviate pain in a subject. Examples of such known analgesic agents include morphine and moxonidine (U.S. Pat. No. 6,117,879).

In addition to the TRPA1-inhibiting compound, the composition can also contain carriers, excipients and additives or auxiliaries. Pharmaceutically acceptable carrier preparations for parenteral administration include sterile or aqueous or non-aqueous solutions, suspensions, and emulsions. Examples of non-aqueous solvents are propylene glycol, polyethylene glycol, vegetable oils such as olive oil, and injectable organic esters such as ethyl oleate. Carriers for occlusive dressings can be used to increase skin permeability and enhance antigen absorption. Liquid dosage forms for oral administration may generally comprise a liposome solution containing the liquid dosage form. Suitable solid or liquid pharmaceutical preparation forms are, for example, granules, powders, tablets, coated tablets, (micro)capsules, suppositories, syrups, emulsions, suspensions, creams, aerosols, drops or injectable solution in ampule form and also preparations with protracted release of active compounds. To these preparations can be added excipients and additives and/or auxiliaries such as disintegrants, binders, coating agents, swelling agents, lubricants, flavorings, sweeteners and elixirs containing inert diluents commonly used in the art, such as purified water.

Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 18th ed., Mack Publishing Co. (1990). The pH and exact concentration of the various components of the pharmaceutical composition are adjusted according to routine skills in the art. See Goodman and Gilman's The Pharmacological Basis for Therapeutics, 10^{th} ed., McGraw-Hill Professional (2001).

Pharmaceutical composition containing a TRPA1-inhibiting compound can be administered locally or systemically in a therapeutically effective amount or dose. They can be administered parenterally, enterically, by injection, rapid infusion, nasopharyngeal absorption, dermal absorption, rectally and orally. An effective amount of a TRPA1-inhibiting compound is an amount that is sufficient to reduce or inhibit a nociceptive pain or a nociceptive response in a subject. For a given TRPA1-inhibitor compound, one skilled in the art can easily identify the effective amount of an agent that modulates a nociceptive response by using routinely practiced pharmaceutical methods. Typically, dosages used in vitro may provide useful guidance in the amounts useful for in situ administration of the pharmaceutical composition, and animal models may be used to determine effective dosages for treatment of particular disorders. Various considerations are described, e.g., in Langer, Science, 249:1527, (1990); Gilman et al. (eds.) (1990), each of which is herein incorporated by reference.

### EXAMPLES

The following examples are offered to illustrate, but not to limit the present invention.

### Example 1

### TRPA1 is Activated by Cinnamaldehyde and Other Sensory Compounds

Since TRPA1 marks neurons that can respond to both heat and cold stimuli, the sensory quality that TRPA1 activation conveys is crucial in understanding the coding of noxious temperature (Story et al., Cell 112, 819-829, 2003). We searched for pharmocological activators of TRPA1. We focused on compounds mostly derived from food items used in oral care and confectionery products that have a sensory component distinct from taste and smell. The list included a number of compounds that signal a cooling or a burning sensation. Using a Fluorometric Imaging Plate Reader (FLIPR), we showed that mouse TRPA1-expressing CHO cells (mTRPA1) show a sharp increase in intracellular calcium upon application of eugenol, gingerol, methyl salicylate, allyl isothiocyanate and cinnamaldehyde. All these compounds are known to cause a pungent burning sensation in humans.

We then tested these compounds against TRPM8 and TRPV1. Only allyl isothiocyanate and cinnamaldehyde were specific to mTRPA1, indicating that the burning sensation that these compounds cause is independent of TRPV1. 600 μM of Methyl salicylate (MeS) was also specific to mTRPA1. However, 2 mM MeS activated TRPV1-expressing cells, corresponding to 25% of the TRPV1 response observed from saturating amounts of capsaicin. Compounds with cooling properties such as spearmint did not activate mTRPA1. Instead, these cooling compounds activated TRPM8, suggesting a similar mode of action to menthol.

We focused on the two mTRPA1-specific compounds. Using FLIPR, we determined the concentration for half maximal activation to be 61±9 μM for cinnamaldehyde and 22±3 μM for allyl isothiocyanate. We also performed ratiometric calcium imaging of CHO cells expressing mTRPA1 and recorded a robust increase in intracellular free calcium upon application of cinnamaldehyde and allyl isothiocyanate. The results show that increasing the cinnamaldehyde concentration dramatically shortened the latency and increased the magnitude of response of mTRPA1-expressing cells. Ruthenium red, a known blocker of mTRPA1, blocked the cinnamaldehyde response. The results also show that cinnamaldehyde and allyl isothiocyanate did not activate TRPV1-, TRPV4-, and TRPM8-expressing CHO cells.

We also characterized the cinnamaldehyde-induced current in mTRPA1 expressing CHO cells. In a whole cell patch clamp setup, cinnamaldehyde elicited a robust desensitized current. The current-voltage (IV) relationship in response to cinnamaldehyde and cold were identical, indicating that both activate the same ion channel. Expression of either mouse or human TRPA1 (hTRPA1) in Xenopus oocytes rendered these cells responsive to cinnamaldehyde as well as to cold temperatures. Interestingly, while the cold-activated current showed a rapid desensitization during the cold pulse, the cinnamaldehyde-activated current was sustained for the full duration of the application. Indeed, the cinnamaldehyde-induced current from mTRPA1-expressing oocytes rarely returned to baseline, even after washing out the compound. Oocytes expressing dTRPA1 (dANTKM1), the Drosophila melanogaster ortholog of mTRPA1, did not respond to cinnamaldehyde.

Repeated applications of cinnamaldehyde to hTRPA1-expressing oocytes showed strong sensitization, in contrast to the desensitizing effect of cold. The second cinnamaldehyde pulse resulted in a current that was on average 250±23% (n=6) of the first pulse, compared to 62±7% (n=5) for the second of two cold pulses. Increased currents by repeated application of cinnamaldehyde (sensitization) in oocyte recordings is in contrast to strong desensitization to cinnamaldehyde observed in mTRPA1-expressing CHO cell. Cold-activated currents, on the other hand, exhibit desensitization in both systems (Story et al., Cell 112, 819-829, 2003).

### Example 2

### TRPA1 is Activated by Bradykinin

Activation of TRPA1 by pungent natural products suggests a nociceptive role for TRPA1. We investigated whether TRPA1 is activated by endogenous noxious chemicals. Bradykinin (BK) is among the most potent algogenic substances released from tissue injury and inflammation. BK directly excites polymodal nociceptors, resulting in an acute painful perception, and further sensitizes these nerves to thermal, chemical, and mechanical stimuli.

Little is known about the mechanism by which BK causes acute excitation of sensory neurons. Bradykinin receptor (B2R), similar to TRPA1, is expressed in a subpopulation of capsaicin-responsive nociceptors. We therefore examined whether TRPA1 is functionally coupled to B2R signaling. Whole cell recording of mTRPA1-expressing CHO cells transiently transfected with B2R showed an acute and immediate current responses to 1 μM BK (n=5). No significant current was observed during BK application in control cells: CHO cells (n=8), TRPA1 cells (n=7), and B2R-only expressing cells (n=6). The currents evoked by BK, cold, and cinamaldehyde have identical reversal potentials and rectification properties, arguing that BK-activated currents are due to TRPA1 activation.

### Example 3

### Role of Phospholipase C, OAG, and Arachidonic Acid in TRPA1 Activation

Phospholipase C (PLC) and phospholipase A2 are activated by BK signaling. Since many TRP channels are modulated by PLC activity, we tested whether downstream affectors of PLC can modulate TRPA1 function. One of the major consequences of PLC activation is the release of calcium from intracellular stores. We therefore tested if passive release of calcium from the stores with the smooth endoplasmic reticulum Ca²⁺-ATPase (SERCA) pump blocker thapsigargin could activate TRPA1 function. We examined the effect of thapsigargin on cells transiently-transfected with hTRPA1 and YFP reporter plasmid. This allowed for a direct comparison between TRPA1-expressing (YFP positive) and control (YFP negative) cells under the same experimental conditions (on the same coverslip). The response in YFP-positive cells was slightly smaller than in YFP-negative cells. Identical results were observed for transiently- and stably-transfected mTRPA1 in CHO cells. Control experiments in which cells were transfected only with the YFP reporter plasmid showed no difference in thapsigargin responses between YFP-positive and -negative cells. Taken together, these results suggest that calcium release does not cause TRPA1 activation.

Another downstream effect of PLC activity is the generation of Diacylglycerol (DAG). Therefore, we tested if l-Oleoyl-2-acetyl-sn-glycerol (OAG, a cell permeable analog of DAG) could activate TRPA1. The results show OAG application yielded a robust response in mTRPA1-expressing CHO cells which could be blocked by ruthenium red. OAG alone gives no response in naïve CHO cells. DAG can be converted to poly-unsaturated fatty acid (PUFAs) such as arachidonic acid (AA). The results also show that AA activated TRPA1-expressing CHO cells, and this activation was blocked by ruthenium red. AA can be converted to numerous metabolites, including prostaglandins. We reasoned that if TRPA1 activation is due to downstream metabolites of AA, then a non-metabolized AA analog would be unable to activate TRPA1. However, such a compound named 5,8,11,14-Eicosatetraynoic acid (ETYA) activated TRPA1- expressing CHO cells. Therefore, AA metabolism is not required for the activation of TRPA1.

TABLE 1
Activation of TRP channels by Sensory Compounds

| | Control | TRPV1 | TRPM8 | TRPA1 | |
|---|---|---|---|---|---|
| Eugenol (Clove oil) Gingerol (Ginger) | —— | ++ | +— | ++ | 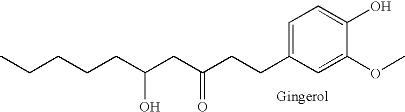 |
| Menthyl lactate Trans-p-menthane-3,8-diol | —— | —— | ++ | —— | 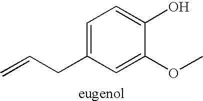 |
| cis-p-menthane-3,8-diol L-carvone (Spearmint oil) WS23 | ——— | ——— | +++ | ——— | 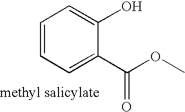 |
| WS3 Isopulegol Methyl salicylate (Wintergreen oil) | ——— | —— —* | ++— | ——+ | 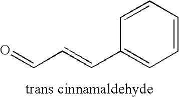 |
| Ally isothiocyanate (Mustard oil) Cinnamaldehyde (Cinnamon oil) | —— | —— | —— | ++ | 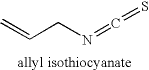 |

Each compound was tested at 600 μM on CHO cells expressing a TRP channel on FLIPR.
*Activation was observed at 2 mM but not at 600 μM.

We next tested if specific inhibition of PLC by U-73122 would affect TRPA1 activation by bradykinin. In calcium imaging studies, BK responses in B2R/TRPA1- expressing CHO cells and B2R-expressing cells were indeed inhibited by 10 μM of U-73122, but not by U-73343 (a similar but inactive analog). We then tested if PLC inhibition of TRPA1 was stimulus specific. U-73122 inhibited TRPA1 activation by cinnamaldehyde and strongly downregulated TRPA1 activation by cold. We further tested the role of PLC inhibition on TRPM8. U-73122 strongly dowregulated the cold- and menthol-induced responses of TRPM8. Preincubation of U-73 122 was necessary to observe a block of the menthol response, suggesting that this compound is not acting as an ion channel blocker.

### Example 4

### Cinnamaldehyde and Bradykinin Activate TRPA1-like DRG Neurons

It has been shown that two distinct populations of cold-responding neurons are present in cultured DRGs. One population is activated by mild cool temperatures and responds to menthol. The other population is activated by colder temperatures and responds to capsaicin but not to menthol. In vivo, TRPA1 is expressed in a subset of TRPV1 neurons, but is not co-expressed with TRPM8. Therefore, we had hypothesized that TRPM8 and TRPA1 mark the two cold populations, respectively.

To further test this hypothesis, and to find out if the pungent compounds described above activate TRPA1 specifically, we performed calcium imaging of adult rat DRG neurons in response to cold, menthol, cinnamaldehyde, allyl isothiocyanate, and bradykinin. The data indicate that cinnamaldehyde activated 39% of cold-activated DRG neurons, but only 1% of cold-insensitive neurons. To a large extent, cinnamaldehyde and menthol activated mutually exclusive populations of cold-responsive neurons, as our model would predict. Allyl isothiocyanate appeared less specific as it activated 63% of the cold-responsive population (including a large overlap with menthol) and 12% of the cold-insensitive population. We have used 1.6 times the EC50 values of cinnamaldehyde and allyl isothiocyanate to enable us to directly compare DRG response profiles to these compounds. Furthermore, raising the concentration of cinnamaldehyde to 200 μM did not show any dramatic shift in response profiles. There were no significant differences in profiles of cinnamaldehyde and allyl isothiocyanate between cultures in the presence of 1 and 100 ng/ml of NGF.

In addition to the pungent sensory compounds, we have shown that BK can activate TRPA1. To demonstrate if this interaction could be physiologically relevant, we tested if BK- and cinnamaldehyde-responsive profiles overlap in cultured DRG neurons. The results show that BK activated 14% (19/138) of total DRG neurons, and 78% (7/9) of cinnamaldehyde-responsive neurons. These results suggest that a majority of TRPA1-expressing neurons also express bradykinin receptors.

### Example 5

### Cinnamaldehyde Causes Nociceptive Behavior in Mice

To provide evidence for the role of TRPA1 in pain signaling, we performed intraplantar injections of mice with cinnamaldehyde (the most specific TRPA1 agonist) and recorded nociceptive behavior. We used capsaicin as a positive control for these experiments. We found that cinnamaldehyde activates TRPA1 with an EC50 of ˜61 μM, while capsaicin is known to activate TRPV1 with an EC50 of 0.7 pM. Around 0.33-10 mM (1-30 μg) of capsaicin has been used in the art for intraplantar injection in rodents (Caterina et al., Science 288: 306-313, 2000). We used two concentrations of cinnamaldehyde injections: 5 mM and 16.4 mM (6.6 and 21.7%g). To identify a negative control more meaningful than vehicle injections, we screened for cinnamaldehyde-like compounds that did not activate TRPA1 in heterologous expression systems. Cinnamic acid, a close analog of cinnamaldehyde, did not activate mTRPA1 even in millimolar concentrations.

Both concentrations of cinnamaldehyde induced licking and shaking of the injected hindpaw during the five minutes assayed post-injection, a behavior not observed in vehicle- and cinnamic acid-injected mice. As expected, TRPV1^{-/-}mice also responded to cinnamaldehyde injections. Interestingly, the response appeared more robust compared to wildtype; however, this difference did not achieve statistical significant (p=0.08).

To provide further evidence of the role of TRPA1 in pain sensation, we investigated if cinnamaldehyde injections could lead to hyperalgesia, an increased response to pain due to sensitization often caused by inflammation or injury. Thirty minutes after cinnamaldehyde injections, paw withdraw latency was significantly lowered in the injected compared to the control paws (p=0.009). In contrast, no significant difference could be observed between the latencies of the two paws when vehicle was injected.

Hyperalgesia to heat is thought to involve sensitization of TRPV1. Despite the robust acute pain behavior of cinnamaldehyde-injected TRPV1^{-/-} mice, our data show that heat hyperalgesia was absent in these mice. Note that paw withdrawal latencies to heat are higher in TRPV1^{-/-} mice, consistent with the partial heat sensitivity phenotype of these mice (Caterina et al., Science 288: 306-313, 2000; and Davis et al., Nature 405: 183-187, 2000). Therefore, the acute nociceptive response of cinnamaldehyde is independent of TRPV1, while the heat hyperalgesia is mediated through TRPV1.

### Example 6

### Allicin is the Chemical in Garlic that is Responsible for TRPA1 Activation

Allicin (diallyl disulfide oxide) is an unstable molecule produced enzymatically from alliin (S-2-propenyl-L-cysteine sulfoxide) when garlic is damaged or cut. We found that addition of allicin to TRPA1- and TRPV1-expressing CHO cells showed an immediate and strong calcium response, similar to the responses to garlic extract. This suggests that allicin might be the main pungent constituent of fresh garlic. We also examined dose response curves for allicin on mTRPA1 and hTRPA1 by FLIPR. The EC50s calculated for mTRPA1 and hTRPA1 are 1.32 μM and 1.91 μM, respectively. In electrophysiological recording experiments, 1 μM allicin was able to activate TRPA1-expressing oocytes at a concentration of 1 μM, consistent with calcium imaging experiments.

To test if garlic extracts and allicin specifically activate TRPA1 in native neurons, we performed calcium imaging of adult rat DRG neurons. We used capsaicin and cinnamaldehyde to mark TRPV1-expressing neurons. Addition of allicin or garlic extract to cultured rat DRG neurons activated a specific population of neurons. High concentrations of garlic extract or allicin (a dilution of 1:50 for garlic, and 100 μM allicin) activated the majority of capsaicin-sensitive DRG neurons. On the other hand, low concentrations of garlic extract and allicin (a dilution of 1:500 for garlic, and 10 μM allicin) activated only the cinnamaldehyde-sensitive neurons (a smaller subset of capsaicin-sensitive population). Importantly, capsaicin-insensitive neurons never responded to garlic extract or allicin.

It is understood that the examples and embodiments described herein are for illustrative purposes only and that various modifications or changes in light thereof will be suggested to persons skilled in the art and are to be included within the spirit and purview of this application and scope of the appended claims. Although any methods and materials similar or equivalent to those described herein can be used in the practice or testing of the present invention, the preferred methods and materials are described.

All publications, GenBank sequences, ATCC deposits, patents and patent applications cited herein are hereby expressly incorporated by reference in their entirety and for all purposes as if each is individually so denoted.

We claim:

1. A method for identifying an inhibitor of noxious cold ion channel TRPA1, comprising (a) contacting a TRPA1 polypeptide with test agents in the presence of a TRPA1 agonist; and (b) identifying a modulating agent that suppresses or reduces a signaling activity of the TRPA1 polypeptide relative to the activity of the TRPA1 polypeptide in the absence of the test agent; thereby identifying a TRPA1 inhibitor; wherein the TRPA1 agonist is selected from the group consisting of cinnamaldehyde, eugenol, gingerol, methyl salicylate, and allicin.

2. The method of claim 1, wherein the TRPA1 agonist is put into contact with the TRPA1 polypeptide prior to contacting the TRPA1 polypeptide with the test agents.

3. The method of claim 1, wherein the TRPA1 polypeptide is human TRPA1 or mouse TRPA1.

4. The method of claim 1, wherein the TRPA1 polypeptide is present in a TRPA1-expressing cell or a cultured neuron.

5. The method of claim 4, wherein the cultured neuron is a cultured DRG neuron.

6. The method of claim 4, wherein the cell is a TRPA1-expressing CHO cell or a TRPA1-expressing Xenopus oocyte.

7. The method of claim 4, wherein the signaling activity is calcium influx into the TRPA1-expressing cell or the cultured neuron.

8. The method of claim 4, wherein the signaling activity is increased intracellular free calcium level of the TRPA1-expressing cell or the cultured neuron.

9. A method for identifying an agent that modulates noxious cold ion channel TRPA1, comprising:
(a) assaying a biological activity of a phospholipase C (PLC) polypeptide in the presence of a test agent to identify one or more modulating agents that modulate the biological activity of the PLC polypeptide; and
(b) testing one or more of the modulating agents for ability to modulate a signaling activity of TRPA1.

10. The method of claim 9, wherein the PLC polypeptide is a PLC isoform that is expressed in dorsal root ganglia (DRG) neurons that express TRPA1.

11. The method of claim 9, wherein the modulating agents inhibit the signaling activity of TRPA1.

12. The method of claim 9, wherein the modulating agents activate the signaling activity of TRPA1.

13. The method of claim 9, wherein (b) comprises testing the modulating agents for ability to modulate calcium influx of a TRPA1-expressing cell.

14. The method of claim 13, wherein the cell is contacted with a TRPA1 agonist to activate TRPA1.

15. The method of claim 13, wherein the cell is a TRPA1-expressing CHO cell or a TRPA1-expressing Xenopus oocyte.

16. The method of claim 13, wherein the cell is a cultured DRG neuron.

17. The method of claim 13, wherein the TRPA1 is human TRPA1 or mouse TRPA1.

18. The method of claim 9, wherein the biological activity is an enzymatic activity of the PLC polypeptide.

19. The method of claim 18, wherein the enzymatic activity is breakdown of phosphatidylinositol-4,5-bisphosphate (PIP2) into diacylglycerol (DAG) and inositol triphosphate (IP3).

20. The method of claim 19, wherein the modulating agents inhibit breakdown of phosphatidylinositol-4,5-bisphosphate (PIP2) into diacylglycerol (DAG) and inositol triphosphate (IP3).

21. A method for identifying a TRPA1 activator with improved properties, comprising:
(a) synthesizing one or more structural analogs of a TRPA1 agonist;
(b) performing a functional assay on analogs to identify an analog that has an improved biological or pharmaceutical property relative to that of the TRPA1 agonist;
thereby identifying a TRPA1 activator with improved properties;
wherein the TRPA1 agonist is selected from the group consisting of cinnamaldehyde, eugenol, gingerol, methyl salicylate, and allicin.

22. The method of claim 21, wherein the improved biological or pharmaceutical property is an enhanced binding affinity for TRPA1.

23. The method of claim 21, wherein the improved biological or pharmaceutical property is an increased ability to penetrate the skins.

24. The method of claim 23, wherein the TRPA1 agonist is cinnamaldehyde.

25. A method for stimulating sensory perception in a subject, comprising (a) providing a subject that contains noxious cold-activated ion channel TRPA1, and (b) administering to the subject a pharmaceutical composition comprising an effective amount of a compound selected from the group consisting of eugenol, gingerol, methyl salicylate, allicin, and cinnamaldehyde; thereby stimulating noxious cold sensation in the subject.

26. The method of claim 25, wherein the compound activates TRPA1.

27. The method of claim 25, wherein the subject is a human.

28. The method of claim 25, wherein the compound is administered to the subject as a food additive.
